(19) United States
(12) Patent Application Publication
(10) Pub. No.: US 2026/0083422 A1
(43) Pub. Date: Mar. 26, 2026

(54) METHOD AND APPARATUS FOR PROCESSING A SERIES OF MEDICAL X-RAY BASED IMAGE RECORDINGS

(71) Applicant: Siemens Healthineers AG, Forchheim (DE)

(72) Inventors: Hendrik DITT, Neustadt an der Aisch (DE); Oliver TAUBMANN, Weilersbach (DE)

(73) Assignee: Siemens Healthineers AG, Forchheim (DE)

(21) Appl. No.: 19/339,739

(22) Filed: Sep. 25, 2025

(30) Foreign Application Priority Data
Sep. 25, 2024 (DE) 10 2024 209 239.8

(51) Int. Cl. A61B 6/00; A61B 6/03; A61B 6/50

(52) U.S. Cl. CPC A61B 6/5235; A61B 6/032; A61B 6/501; A61B 6/504; A61B 6/507; A61B 6/5217; A61B 6/5258; A61B 6/54; A61B 6/481

(57) ABSTRACT

A method for processing a series of medical X-ray based image recordings, comprises: selecting a series of successive relevant images from a series of image recordings; examining image elements of the successive relevant images for a maximum image value; defining an initial image from the successive relevant images; generating a final image in which the image elements whose maximum image is the initial image are represented in a different color from the image elements whose maximum image is a relevant image recorded after the initial image and/or from the image elements whose image values lie within a specified range of variation in all relevant images; and outputting the final image.

### CROSS-REFERENCE TO RELATED APPLICATION(S)

The present application claims priority under 35 U.S.C. § 119 to German Patent Application No. 10 2024 209 239.8, filed Sep. 25, 2024, the entire contents of which is incorporated herein by reference.

### FIELD

One or more example embodiments of the present invention relate to a method and an apparatus for processing a series of medical X-ray based image recordings, to a control facility for controlling a medical-engineering imaging system, in particular a CT system, and to a medical-engineering imaging system.

One or more example embodiments of the present invention are set in the field of image data analysis of medical image data, which is used to create data-based representations of organs or tissues, in particular for the visual representation and/or for the functional representation of organs or tissues.

### BACKGROUND

In the case of an acute stroke, a plurality of imaging examinations (scans) have to be carried out in order to ascertain the cause and the severity of the stroke. Typically a computed tomography (CT) scan is firstly carried out without contrast agent in order to find out whether the patient has a hemorrhage, and in order to look for early signs of a stroke (for example, signs of a hyperdense media). If it is not possible to discern a hemorrhage, a CT angiography (CTA) is carried out, followed by a perfusion CT. With a CTA it is possible to localize the clot, while the perfusion CT provides insight on the severity of the stroke and answers the question of whether regions of tissue are still present which can be saved by a therapy.

Contrast agent-enhanced imaging data is frequently used which was obtained with X-ray computed tomography (CT) using contrast agents. Contrast agents for the X-ray CT are frequently iodine-containing contrast agents. This is useful for highlighting structures such as blood vessels which would otherwise be difficult to distinguish from their surroundings. Often images are recorded with as well as without X-ray contrast. The use of contrast agent can also help to obtain, functional items of information about tissue, for example items of perfusion information, i.e. information about the level of the blood supply of a region of tissue.

Perfusion CT thus supplies important data, in particular if the occurrence of the stroke was not that long ago. In practice, it represents the most used possibility by far for determining the infarct core as well as the penumbra.

Unfortunately perfusion CT has some drawbacks, however. Thus, inter alia, an additional administration of contrast agent (after a first administration of contrast agent for the CTA), the lack of opportunity to use bolus triggering (a baseline is always needed for calculating the perfusion) or also the relatively high exposure to radiation due to recording over a relatively long period.

In the first six hours after the occurrence of the symptoms of a stroke it is sufficient to gather an unenhanced recording as well as a CTA of the patient. Only when more time has elapsed are further examinations necessary. In many cases a perfusion recording is still made even within the first six hours since this allows the best insight into the processes in the brain. In many cases just one multi-phase CTA is also used (as a rule comprising three phases) whose first phase is scanned with the aid of bolus triggering precisely at the instant of the arterial contrast agent enhancement. This first phase comprises the aortic arch through to the calvarium, the following phases in the interval of 7-10 seconds record only the brain itself respectively. These recordings are normally evaluated by simply looking at the images side by side. The instant of maximum enrichment of the contrast agent provides insight about the condition of the patient. It is also possible to calculate “quasi perfusion results” from this data but, owing to the lack of a (contrast agent-free but otherwise equivalently recorded) baseline dataset, the short-duration sampling and the possible lack of the instant of maximum enhancement, this data can only represent an approximation.

Further, it is possible to represent the inflow of the contrast agent over time in color. Vessels, which have the highest contrast agent level in the first recording, are represented, for example, in red, those in the second image in green and in the last image in blue. However, only the vessels are represented (similar to the CT flow visualization) and it is also not possible to draw conclusions about regions which have no enhancement.

### SUMMARY

It is an object of one or more example embodiments of the present invention to disclose a method and an apparatus for processing a series of medical X-ray based image recordings, which a control facility (also referred to as a control device) for controlling a medical-engineering imaging system, in particular a CT system and a medical-engineering imaging system, with which the above-described drawbacks can be avoided. In particular, it is an object of one or more example embodiments of the present invention to process CTA recordings such that a doctor can use them more effectively to examine a stroke.

At least this object is achieved by a method, an apparatus, a control facility and a medical-engineering imaging system as claimed in the independent claims.

An inventive method serves for processing a series of medical X-ray based image recordings, in particular a CTA, which have been recorded from a patient, after an administration of contrast agent, in intervals during the inflow of the contrast agent. The method comprises the following steps:
providing the series of image recordings, and selecting a series of successive relevant images from these image recordings,
examining the image elements of the relevant images for a maximum image value, wherein when an image element is examined, the value of this image element is considered at the same image coordinate in all relevant images, and the image in which the value of this image element is maximal is identified as the maximum image for this image element,
defining an initial image (S) from the relevant images,
generating a final image (E) in which the image elements whose maximum image is the initial image are represented in a different color to the image elements whose maximum image is a different relevant image, which has been recorded after the initial image and/or as the image elements (V) whose image values lie within a specified range of variation in all relevant images,
outputting the final image.

The inventive method is preferably a computer-implemented method.

The series of image recordings can be provided by a provision of corresponding image data. The image data can take place by way of an imaging device, an image data storage device, a network with access to stored image data or any other data-based provision.

The final image can be output to an output unit (for example a screen) and/or in a data-based manner, for example by way of storage in a data store or by being made available in a network.

The method is particularly advantageous for supplementing CTA images for the examination of a stroke. However, other advantageous applications are also possible in which a series of medical X-ray based image recordings, which have been recorded from a patient (human or animal), after an administration of contrast agent, in intervals during the inflow of the contrast agent, are to be processed. This processing comprises at least one highlighting in color of regions of these images.

Firstly, the series of image recordings is provided. This can be done in that images are recorded or are downloaded from a database. The images are preferably CT images, in particular from a CTA, but could also be MRI images.

A series of successive relevant images is selected from these image recordings. It is possible for all or only some of these relevant images to be image recordings. Therefore, the image recordings can certainly be a 3D image stack (i.e. a three-dimensional recording of a region of interest (ROI) and the series of relevant images can comprise a sectional image (at the same coordinate respectively) of the respective image recordings. In addition, it is possible to select images from the image recordings from a smaller time interval. Hereinafter, for a simple understanding it can be assumed that an exemplary series of three to five CT image recordings exists and the relevant images from each image recording comprise a 2D sectional image which all show the same region of a brain.

If the relevant images exist, their image elements (i.e. basically pixels or voxels) are examined for a maximum image value. The same image coordinate is always considered for an image element, i.e. basically the same position of the recorded organ. For each examined image element it is the image which represents the maximum of the value of this image point which is ascertained as the maximum image. It should be noted in this connection that, as a rule, a noise is irrelevant. Only a value which can clearly be recognized as a maximum should count as the maximum, and this will be explained in more detail below. There can certainly be image elements which do not have a maximum and instead lie at most in a (small) value range. These can represent a parenchyma and will be considered in more detail below.

Irrelevant regions, such as background or bones, should be excluded from this examination. This can be done by defining a mask before the examination, which mask excludes regions which should not be examined. However, an image segmentation can also take place and only image elements which belong to a specified segment are examined. Such techniques are known in the prior art. If relevant images of a brain exist therefore and a stroke is to be examined, then it is advantageous to remove the cranial bone as well as the background. The ventricles and the atrium can also be excluded from the examination.

In practice the gray-scale values of the individual voxels (image elements) of slice images can be examined for a maximum brightness for this purpose. Preferably, all image elements which are to be examined are firstly gone through systematically and particularly preferably in this context, a mask is created in which the number of the maximum image of the series is entered for each image point. It is also possible to make an entry if there was no maximum, for example the value “0”. This mask can then be used for the subsequent method steps. This has the advantage that the voxels only have to be systematically gone through once. In anticipation it should be stated that the mask can also have items of information about the values of voxels of preceding and subsequent images, for example in the form of a vector (pre-value, number of maximum image, post-value).

For each examined image point it is therefore at least known which of the images is the maximum image, or possibly that this image point does not have a maximum.

Next, an initial image is defined from the relevant images. In general, the initial image should show the first contrast agent maximum, and, as a rule, is interpreted as a supply with arterial blood. In the brain, healthy regions, for example, are characterized thereby. In the case of the liver, regions which have an arterial blood supply can be interpreted as diseased regions, however. The initial image can be selected in a plurality of ways. For example, the entire series of relevant images can already have been chosen such that in the first image they show the first inflow of contrast agent (i.e., as a rule, arterial blood). Then the relevant image which comes first time-wise can simply be selected as the initial image. However, it is also possible to firstly examine in which image of the series of relevant images this maximum is to be found. The relevant image which comes first time-wise and which has been identified as a maximum image for more than one specified number of image elements, can be identified for this. It should be noted in this respect that, statistically, any image element has its maximum in the image which comes first time-wise. In order to exclude such outliers, a minimum number of image elements which should have their maximum in this image should be specified.

It should also be noted that different regions of an organ receive the arterial blood at different times. It is certainly possible to define a plurality of initial images for different regions of the organ in this regard. However, the regions should be firmly specified for this or be ascertained as regions. One possibility will be presented below (comparison of pre-value with post-value) as to how, in respect of a maximum, it is possible to differentiate between an arterial blood supply and a collateral one. This possibility can certainly be considered as an additional check for any image point whose maximum follows the initial image time-wise.

It is therefore accordingly known relative to which initial value time-wise (the initial image is representative of an instant) the maximums of the image values should be considered.

The final image is accordingly generated. This final image can be a relevant image which is modified or it can also be created afresh. Since the subject of the relevant images is to be shown subsequently, the final image should show this subject or it should be possible to lay it over an additional image with the subject (possibly one of the relevant images), so it supplements the first one with color markings.

The image elements whose maximum image is the initial image are represented in a different color in this final image than the image elements whose maximum image is a different relevant image, which has been recorded after the initial image, and/or the image elements whose image values lie within a specified range of variation (for example around a mean) in all relevant images.

The image elements whose maximum image is the initial image (i.e. which show an arterial blood supply) can be represented with normal shades of gray, i.e. unchanged, in particular if the subject is the brain. It is possible to potentially draw on a representation of a different image to the initial image (for example the last image of the series) since the initial image shows the maximum enhancement. If the subject is something other than the brain, for example the liver, these image elements could be marked in color, however, and be represented, in particular, in a warning color (for example red), since they could show, for example, a hemorrhage in the liver.

The image elements whose maximum image is a different relevant image, which has been recorded after the initial image, are preferably (at least if the subject is the brain) marked with a first warning color, for example yellow. As a rule, they show tissue with a collateral blood supply (in the brain, a penumbra). It is particularly advantageous here if the color coding correlates with the place value of the maximum image in the series of the relevant images (the image number), i.e. different colors or hues are used to designate the instant of the maximum. If the subject is something other than the brain, for example the liver, these image elements could be represented in their normal shades of gray, however, since in this case they could also show healthy tissue.

The image elements whose image values lie within a specified range of variation in all relevant images (for example around a mean), do not experience an inflow of contrast agent. Since the contrast agent basically represents the blood supply, it can be assumed that these regions do not have a blood supply, i.e. are seriously damaged (in the brain, a parenchyma). These image elements are preferably marked with a second warning color, for example red.

The final image, which therefore shows (possibly different) color markings and preferably also the subject in gray scales, is then output so it can be reviewed for a diagnosis, for example by a doctor or an examination algorithm. The examination itself is not part of the present invention, the aim of which is to generate the final image.

An inventive apparatus serves for processing a series of medical X-ray based image recordings of a patient, which, after an administration of contrast agent, have been recorded in intervals during the inflow of the contrast agent, in particular according to a method as claimed in one or more example embodiments of the present invention. The apparatus comprises the following components:
a data interface designed to receive the series of image recordings,
   a selection unit designed for selecting a series of successive relevant images from these image recordings,
   an examination unit designed for examining the image elements of the relevant images for a maximum image value, wherein when an image element is examined, the value of this image element is considered at the same image coordinate in all relevant images, and the image in which the value of this image element is maximal is identified as the maximum image for this image element,
   an initial image unit designed for defining an initial image from the relevant images,
   a marking unit designed for generating a final image in which the image elements whose maximum image is the initial image is represented in a different color to the image elements whose maximum image is a different relevant image which has been recorded after the initial image and/or to the image elements whose image values lie within a specified range of variation in all relevant images,
   a data interface designed for outputting the final image.

The function of the components of the apparatus has already been described above. The apparatus is preferably designed to execute an inventive method.

Further embodiments of the inventive apparatus follow directly from the various embodiments of the inventive method, and vice versa. In particular, individual features and corresponding explanations as well as advantages, which refer to the various embodiments of the inventive method, can be transferred analogously to corresponding embodiments of the inventive apparatus. In this case the functional features of the method can be embodied by corresponding units or modules of the system. In particular, the inventive apparatus is conceived or programmed such that it executes the inventive computer-implemented method.

One or more example embodiments of the present invention therefore provide a false color image as a final image, which, for example in the case of a brain examination, can highlight a penumbra and/or a parenchyma in color. With, for example, three image recordings, of which the first is the initial image, for example voxels, which appear brightest in the second image, are represented in yellow and voxels, which appear brightest in the third image, are represented in orange. Voxels, which have the same gray-scale value in all images (possibly within a specified range of variation) are represented in red in order to designate them as the core region of the stroke. Voxels, which have the greatest enhancement in the first recording, are still represented by their HU value on a gray scale.

In contrast to the other approaches in which only the vessels are considered, the vessels as well as the parenchyma are considered in the approach described here. Parenchyma regions, through which a contrasted vessel still runs but which no longer has a blood supply, can consequently be marked in a different color. Regions which no longer have a blood supply, in which it is no longer possible to see a vessel therefore, can also be represented thereby. A representation of the infarct core is thus also possible if it should still not appear in the unenhanced phase.

An inventive control facility serves to control a medical-engineering imaging system, in particular a CT system or a diagnosis system. It comprises an inventive apparatus and/or is designed for carrying out an inventive method.

An inventive medical-engineering imaging system is preferably a CT system (but could also be an MRI system) or a diagnosis system, and comprises an inventive control facility.

One or more example embodiments of the present invention can be implemented, in particular, in the form of a computer unit with suitable software. The computer unit can have for this purpose, for example, one or more collaborating microprocessor(s) or the like. In particular, it can be implemented in the computer unit in the form of suitable software program parts. An implementation largely in terms of software has the advantage that even previously used computer units can be easily retrofitted by way of a software or firmware update in order to work inventively. In this regard the object is also achieved by a corresponding computer program product with a computer program which can be loaded directly into a memory facility of a computing system, with program segments in order to execute the steps of the inventive method when the program is executed in the computer unit. Apart from the computer program, such a computer program product can optionally comprise additional integral parts, such as documentation and/or additional components, also hardware components, such as hardware keys (dongles, etc.) in order to use the software.

A computer-readable medium, for example a memory stick, a hard disk or another transportable or permanently installed data carrier, on which the program segments of the computer program, which can be read in and executed by a computer unit, are stored, can serve for transportation to the computer unit and/or for storage on or in the computer unit.

Further, particularly advantageous embodiments and developments of the present invention can be found in the dependent claims as well as the description below, with it being possible for the claims of one category of claims to also be developed analogously to the claims and parts of the description relating to a different category of claims and, in particular, individual features of different exemplary embodiments or variants to also be combined to form new exemplary embodiments or variants.

According to one preferred embodiment of the method, in the final image, with regard to the image elements whose maximum image has been recorded after the initial image, image elements with different maximum images are represented in a different color. Voxels which appear brightest in a later phase (an image, which has been recorded after the initial image), are preferably represented in different colors or hues (for example yellow/orange or different shades of yellow or a transition from yellow to red) according to their recording instant (the image number in the series).

According to a preferred embodiment of the method, in the final image, with regard to the image elements whose maximum image has been recorded after the initial image, image elements, for which a maximum image has been identified, are represented in a different color to the image elements whose image values lie within a specified range of variation in all relevant images. Image values which do not have a maximum in the series are regarded as not having a blood supply. They can be marked in red, for example, whereas image points whose maximum comes after the initial image are represented in yellow or orange.

According to a preferred embodiment of the method, for image coordinates of the relevant images relative to the respective maximum image, a pre-value of the corresponding image element in a previously recorded image and a post-value of a corresponding image element in a subsequently recorded image are compared with one another. For the image points which are preferred here, a maximum does not lie in the first image. The image, which comes before the maximum in the series image, can thereby be considered for an image point and the value of the corresponding image point can be taken as a “pre-value”. For image points whose maximum does not lie in the last image, a post-value can also be ascertained accordingly (in the image which follows, the maximum image in the series).

In tissue with a collateral blood supply, firstly no contrast agent is visible, then comes the maximum, followed by a drop in the contrast agent level. The pre-image will therefore show less contrast agent (and be darker) than the post-image. The situation is different in tissue which has an arterial blood supply. Here an image point before the maximum will appear brighter than after the maximum. The pre-value will therefore be higher than the post-value in this tissue which has an arterial blood supply.

According to the method, preferably in the case where the pre-value is greater than the post-value, an image element will therefore be associated with tissue which has an arterial blood supply. Alternatively or in addition, preferably in the case where the pre-value is less than the post-value is, this image element is associated with tissue which has a non-arterial, in particular collateral, blood supply. In the final image, tissue which has an arterial blood supply is then preferably marked in a different color to tissue which has a non-arterial blood supply. It can preferably be assumed here that voxels, which have their (significant) maximum in the initial image, always have an arterial blood supply and voxels, which have their (significant) maximum in subsequent images have, as a rule, a collateral blood supply, apart from when in the subsequent image in turn this voxel is darker than in the initial image. In this case the voxel likewise has an arterial blood supply. In this way it is possible to define regions in which the contrast agent inflows only after the initial image and a new, individual initial image can be defined for this region. Different regions can then be marked differently.

It has already been noted above that, in practice, it can be disadvantageous to consider only the highest value of an image point since this can be subjected to noise. A maximum should clearly stand out. Otherwise this image point should be associated with a parenchyma. According to a preferred embodiment of the method, in the course of examination of image elements for a maximum image value therefore, only the image elements whose maximum
overshoots a specified absolute limit value, and/or
overshoots a specified interval from a mean of the values of the image points, and/or
overshoots a specified interval from a mean of the values of the adjacent image points and/or
lies outside of the specified range of variation should be considered.

In addition it can also be advantageous to carry out an image smoothing before the evaluation, or to apply morphological operations. This is known in the prior art and reduces undesirable variations in the image values.

According to a preferred embodiment of the method, the image recordings are images of the brain, preferably CT scans of the brain. It is preferred that the relevant images are sectional images of successive CT scans. It is particularly preferred that image regions for whose image elements a maximum image has been identified, which has been recorded after the initial image and/or which are associated with tissue which has a non-arterial, in particular collateral, blood supply, are regarded as penumbra and/or image elements whose image values lie within a specified range of variation in all relevant images are regarded as a parenchyma.

Preferably, the series of image recordings comprises fewer than ten successive recordings, preferably fewer than five. Basically, three recordings are already sufficient, although the first should be the initial image.

It is preferred that in one embodiment of the method, image values of image elements whose maximum image was recorded after the initial value are compared with image values of image elements whose maximum image is the initial image and a blood flow or a number of blood vessels is estimated therefrom. Therefore the shades of gray of tissue with a collateral blood supply are compared with shades of gray of tissue which has an arterial blood supply.

Sometimes it is very helpful if, in addition to the previously determined information as to when the blood arrives in the case of tissue with a collateral blood supply, it is also known how much blood is arriving compared to the healthy tissue. If the blood arrives late, for example (in the case of a collateral supply), then it certainly makes a difference to the doctor whether this collateral supply manages (at least at the instant of the examination still) to transport a similar volume of blood, and therewith oxygen, into the affected region, or whether hardly any more blood arrives. There are various possibilities for quantifying this volume or the difference. An exemplary possibility for this would be to create an image by averaging all instants, which image then shows how much blood arrives on average. A further possibility would be to create an image which represents the maximum across all instants and thus shows how much blood at most is arriving.

Instead of individual image points, (relatively large) regions with a collateral supply should be considered for this purpose. Averaged (over a region), gray-scale values would therefore be compared with one another between various such regions in order to determine the volume of blood relative to one another. For example, a region of tissue with a collateral blood supply could be compared with a corresponding region of healthy tissue, i.e. which has an arterial blood supply. In particular, a comparison of areas on the left half of the brain with its counterpart on the right half of the brain, and vice versa, is expedient for this since most strokes are pronounced on one side and the brain is otherwise symmetrical for the most part.

Unfortunately, “reference” time phases without any contrast agent are scarcely available in conventional mCTA recordings, however there would be the (optional) possibility here of also taking into account a prior non-contrasted recording which, as a rule, is also recorded. The gray-scale values would differ slightly from those in the CTA because this recording was done differently, but it is completely possible to identify certain regions (for example, gray vs. white substance) on the non-contrasted image more easily. Gray and white regions could then be considered separately in the comparison in order to be able to better separate the gray-scale value differences caused by contrast agent or hemodynamics from the gray-scale value differences caused by a priori different tissue. Alternatively, an atlas registered with the image could be used for determining gray and white substance (also without a non-contrasted image).

It is therefore preferred that the relevant image values are read from different relevant images to the maximum image, preferably in a pre-image.

It is preferred, moreover, that in one embodiment of the method the comparison is carried out
with corresponding areas of the opposite half of the brain respectively or
with areas with corresponding image values or
with areas with corresponding brain substance (gray matter, white matter).

It is preferred that the means of the image values are calculated and compared with one another in the respective areas.

Before determining the phase of the highest enhancement per voxel, a relatively intense smoothing should take place in the image space in order to reduce incorrect associations owing to noise and image artifacts. A method which prevents the signals from (relatively large) vessels, bones, and parenchyma being mixed up is expedient for this. This is possible, for instance, by way of a prior segmentation of bones and vessels and corresponding consideration by masking in the filter kernel or alternatively/in addition, by way of bilateral filters which introduce an additional weighting of the share on the basis of the voxel intensity.

According to a preferred embodiment of the method, a smoothing of the image recordings or the relevant images in the image space is carried out before an examination of the image elements. This has the advantage that incorrect associations owing to noise and image artifacts can be reduced. It is preferred that firstly a segmentation of bones and vessels is carried out, and these are then masked in the filter kernel, and/or that a bilateral filter is used which weights a share of voxels on the basis of a voxel intensity. This embodiment prevents signals from (larger) vessels, bones, and parenchyma being mixed up.

For this purpose, a preferred apparatus comprises a filter unit designed for smoothing image recordings in the image space. It is preferred that the filter unit is designed for segmenting bones and vessels in image recordings and subsequent masking of the segmented regions and filtering of the other regions and/or wherein the filter unit comprises a bilateral filter.

The use of AI-based methods (AI: “Artificial Intelligence”) is preferred for the inventive method. Artificial intelligence is based on the principle of machine-based learning, and, as a rule, is carried out with an adaptive algorithm which has been trained accordingly. The English expression “Machine Learning” is frequently used for machine-based learning, with this also encompassing the principle of “Deep Learning”.

Preferably, components of one or more example embodiments of the present invention are in the form of a “Cloud service”. Such a Cloud service serves to process data, in particular via artificial intelligence, but can also be a service based on conventional algorithms or a service in which an evaluation by humans takes place in the background. In general, a Cloud service (hereinafter also called “Cloud” for short) is an IT infrastructure in which, for example, storage space or computing capacity and/or application software is provided via a network. The user and the Cloud communicate via data interfaces and/or data transfer protocols. In the present case it is particularly preferred that the Cloud service provide computing capacity as well as application software.

In the context of a preferred method, the Cloud service is provided via the network with data which is obtained in the context of one or more example embodiments of the present invention. The Cloud comprises a computing system which, as a rule, does not comprise the local computer of the user. The method can be implemented in a network via a combination of commands. The data calculated in the Cloud is subsequently sent to the local computer of the user again via the network.

### BRIEF DESCRIPTION OF THE DRAWINGS

The present invention will be explained in more detail once again below on the basis of exemplary embodiments and with reference to the accompanying figures. Identical components are provided with identical reference numerals in the various figures. As a rule, the figures are not to scale. In the drawings:

FIG. 1 shows a conventional CT system with an inventive apparatus,

FIG. 2 shows a block diagram of an inventive method,

FIG. 3 shows a series of relevant recordings.

### DETAILED DESCRIPTION

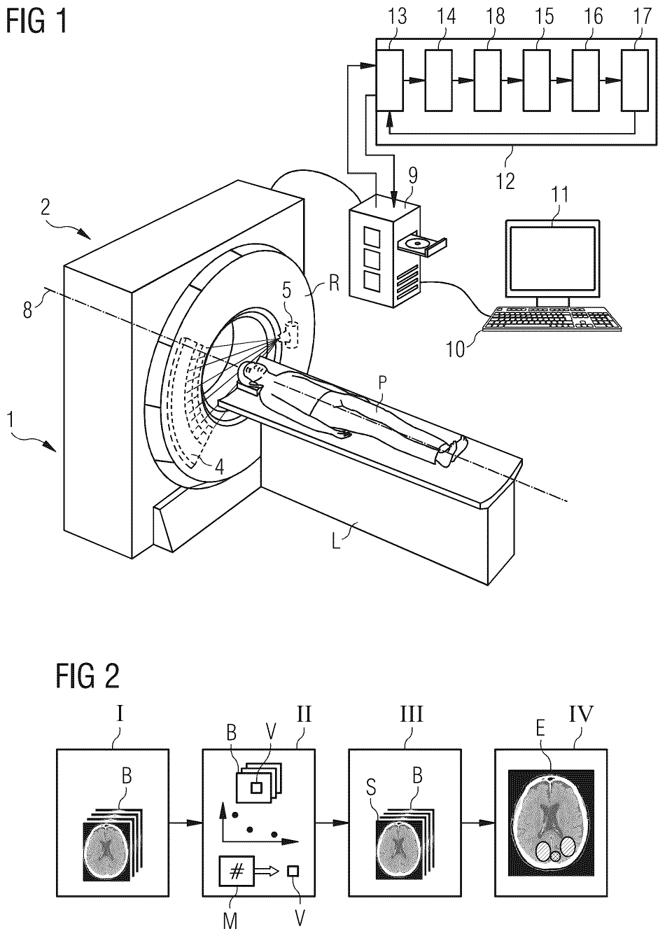

FIG. 1 shows a computed tomography system (CT system) 1 with a radiation detector 4 and an X-ray source 5. The X-ray source 5 is embodied to expose the radiation detector 4 to X-ray radiation. The CT system 1 shown comprises a gantry 2 with a rotor R. The rotor R comprises the X-ray source 5 and the radiation detector 4.

The rotor R can rotate about the axis of rotation 8. The patient P is supported on the patient couch L and can move along the axis of rotation 8 by way of the gantry 2. The computing unit 9 is provided for controlling the CT system 1 and/or for generating an image dataset based on signals detected by the radiation detector 4.

Customarily a (raw) X-ray image dataset of the patient P is recorded from a large number of angle directions via the radiation detector 4. A (final) image dataset can subsequently be reconstructed on the basis of the (raw) X-ray image dataset via a mathematical method, for example comprising a filtered back projection or an iterative reconstruction method.

The computing unit 9 serves here as a control facility 9 for controlling the CT system 1. An input facility 10 and an output facility 11 are connected to this computing unit 9. The input facility 10 and the output facility 11 can make, for example, an interaction by a user or the representation of a generated image dataset B possible.

The control unit comprises an apparatus 12 for inventively processing a series of medical X-ray-based image recordings. Owing to the large number of components, the apparatus 12 was represented above the control facility 9. The apparatus 12 comprises a data interface 13, a selection unit 14, an examination unit 15, an initial image unit 16 and a marking unit 17.

The data interface 13 serves to receive the series of image recordings B and to output the final image E.

The selection unit 14 serves to select a series of successive relevant images B from these image recordings B.

The examination unit 15 serves to examine the image elements V of the relevant images B for a maximum image value, wherein when an image element V is examined, the value of this image element V is considered at the same image coordinate in all relevant images B, and the image in which the value of this image element V is maximal is identified as the maximum image for this image element V.

The initial image unit 16 serves to define the first relevant image time-wise as an initial image S which has been identified as the maximum image for more than one specified number of image elements V.

The marking unit 17 serves to generate a final image E in which the image elements V whose maximum image is the initial image S are represented in a different color to the image elements V whose maximum image is a different relevant image, which has been recorded after the initial image S and/or as the image elements V whose image values B lie within a specified range of variation in all relevant images.

FIG. 2 shows an inventive method for processing a series of medical X-ray based image recordings B.

In step I, the series of image recordings B is provided. In this example these are current recordings of the CT system from FIG. 1. A series of successive relevant images B is selected from the image recordings B, for example sectional images from the same plane.

In step II, the image elements V of the relevant images B are examined for a maximum image value, wherein when an image element V is examined, the value of this image element V is considered at the same image coordinate in all relevant images B, and the image in which the value of this image element V is maximal is identified as the maximum image for this image element V. The gray-scale values of the individual voxels are examined, with a maximum brightness corresponding to a maximum value.

In step III, an initial image S is defined. In this example it is the first relevant image B time-wise, but could also be the first image B which has been identified as the maximum image for more than one specified number of image elements V.

In step IV, a final image E is generated in which the image elements V whose maximum image is the initial image S are represented in a different color to the image elements V whose maximum image is a different relevant image, which has been recorded after the initial image S and/or as the image elements V whose image values lie within a specified range of variation in all relevant images B. This final image is then output.

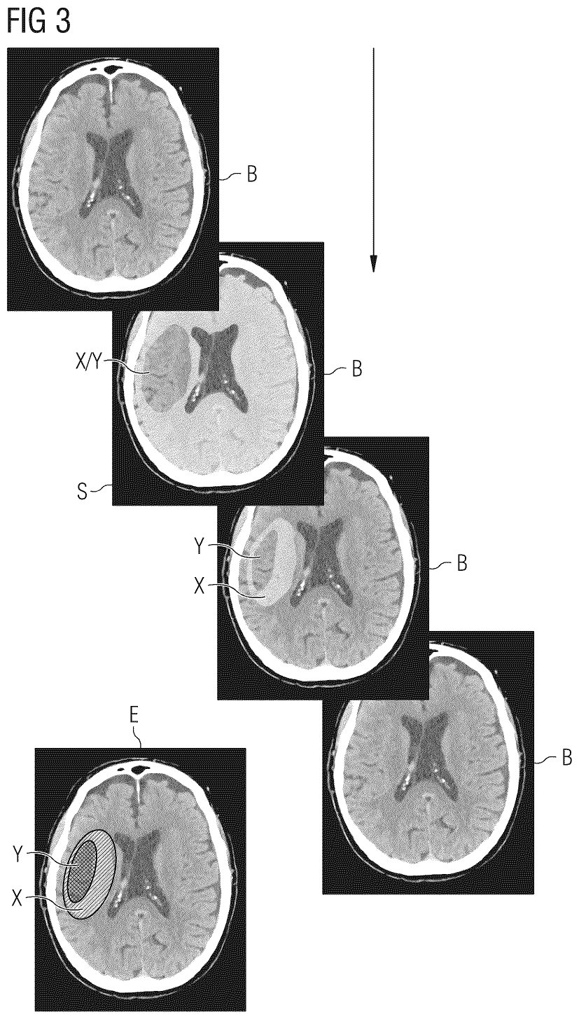

FIG. 3 shows a series of relevant image recordings B. Here a series of four images B is represented whose recording time runs from top to bottom. These are now evaluated by the inventive method and a final image E (bottom left) generated. These images B are merely intended to provide an understanding of embodiments of the present invention by way of example.

In this case, no maximum inflow of the contrast agent has taken place yet in the first image B and therefore the second image B is used as the initial image S. It can be seen here that contrast agent has not yet flowed through a region in the left half of the brain. It is still not clear which part is the penumbra X and which the parenchyma Y.

In the second image B, it can be seen that the contrast agent has disappeared again in most of the brain. However, a part of the region which was dark is now light, and this argues in favor of a collateral blood supply (penumbra X).

In the last image B, the contrast agent has disappeared completely. It is now clear that the dark region in the third image B is a parenchyma Y since at no time did contrast agent arrive there. In the final image, parenchyma Y and penumbra X are marked in different colors.

In conclusion, it will be pointed out once again that the present invention described in detail above are merely exemplary embodiments which can be modified in a wide variety of ways by a person skilled in the art without departing from the scope of the present invention. Furthermore, use of the indefinite article “a” or “an” does not preclude the relevant features from also being present multiple times. Similarly, the term “unit” does not preclude the relevant components from being composed of a plurality of cooperating sub-components which can possibly also be spatially distributed. The term “a number” should be read as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for processing a series of medical X-ray based image recordings recorded from a patient, after an administration of contrast agent, in intervals during an inflow of the contrast agent, the method comprising:
providing the series of medical X-ray based image recordings;
selecting a series of successive relevant images from the medical X-ray based image recordings;
examining image elements of the successive relevant images for a maximum image value, wherein when an image element is examined, a value of the image element is considered at a same image coordinate in all relevant images, and an image in which a value of the image element is at a maximum is identified as a maximum image for the image element;
defining an initial image from the successive relevant images;
generating a final image in which image elements for which the maximum image is the initial image are represented in a different color from at least one of the image elements for which the maximum image is a different relevant image recorded after the initial image, or the image elements having image values that lie within a specified range of variation in all relevant images; and
outputting the final image.

2. The method as claimed in claim 1, wherein, in the final image, with regard to the image elements for which the maximum image has been recorded after the initial image, at least one of the image elements with different maximum images are represented in a different color, or image elements for which the maximum image has been identified, are represented in a different color from the image elements where image values lie within the specified range of variation in all relevant images.

3. The method as claimed in claim 1, wherein, for image coordinates of the relevant images relative to a respective maximum image, a pre-value of a corresponding image element in a previously recorded image and a post-value of a corresponding image element in a subsequently recorded image are compared with one another.

4. The method as claimed in claim 1, further comprising:
considering, during a course of examination of the image elements for a maximum image value, only image elements that at least one of
overshoots a specified absolute limit value,
overshoots a specified interval from a mean of values of image points,
overshoots a specified interval from a mean of values of adjacent image points, or
lies outside of the specified range of variation.

5. The method as claimed in claim 1, wherein the medical X-ray based image recordings are images of a brain.

6. The method as claimed in claim 1, wherein the series of medical X-ray based image recordings includes fewer than ten successive recordings.

7. The method as claimed in claim 1, further comprising:
comparing image values of image elements for which a maximum image was recorded after the initial image with image values of image elements where a maximum image is the initial image; and
estimating a blood flow or a number of blood vessels is based on the comparing.

8. The method as claimed in claim 7, wherein the comparing is carried out with
corresponding areas of an opposite half of a brain, respectively,
areas with corresponding image values, or
areas with corresponding brain substance, and wherein
means of the image values are calculated and compared with one another in respective areas.

9. The method as claimed in claim 1, further comprising:
before examination of the image elements, smoothing the medical X-ray based image recordings or the relevant images in an image space, wherein
firstly, a segmentation of bones and vessels is carried out, and the segmented bones and vessels are then at least one of masked in a filter kernel, or a bilateral filter is used which weights a share of voxels based on a voxel intensity.

10. An apparatus for processing a series of medical X-ray based image recordings recorded from a patient after an administration of contrast agent, in intervals during an inflow of the contrast agent, according to the method as claimed claim 1, the apparatus comprising:
a data interface configured to receive the series of medical X-ray based image recordings;
a selection unit configured to select the series of successive relevant images from the medical X-ray based image recordings;
an examination unit configured to examine the image elements of the successive relevant images for a maximum image value, wherein
when an image element is examined, a value of the image element is considered at a same image coordinate in all relevant images, and an image in which a value of the image element is at a maximum is identified as a maximum image for the image element;
an initial image unit configured to define an initial image from the successive relevant images;
a marking unit configured to generate a final image in which image elements for which the maximum image is the initial image are represented in a different color from at least one of the image elements for which the maximum image is a different relevant image recorded after the initial image, or the image elements with image values lying within a specified range of variation in all relevant images;
a data interface configured to output the final image.

11. The apparatus as claimed in claim 10, further comprising:
a filter unit configured to smooth image recordings in an image space.

12. A control device to control a medical-engineering imaging system, the control device comprising the apparatus as claimed in claim 10.

13. A medical-engineering imaging system comprising the control device as claimed in claim 12.

14. A non-transitory computer program product, comprising commands that, when executed by a computer, cause the computer to execute the method as claimed in claim 1.

15. A non-transitory computer-readable storage medium, comprising commands that, when executed by a computer, cause the computer to execute the method as claimed in claim 1.

16. The method as claimed in claim 3, wherein
in response to the pre-value being greater than the post-value, the corresponding image element is associated with tissue which has an arterial blood supply,
in response to the pre-value being smaller than the post-value, the corresponding image element is associated with tissue which has a non-arterial blood supply, and
in the final image, tissue which has arterial blood supply is marked in a different color from tissue which has a non-arterial blood supply.

17. The method as claimed in claim 16, wherein the non-arterial blood supply is a collateral blood supply.

18. The method of claim 5, wherein the images of the brain include CT scans of the brain.

19. The method of claim 18, wherein at least one of
the successive relevant images are sectional images of successive CT scans,
image regions for image elements where at least one of a maximum image has been identified, the image elements have been recorded after the initial image or the image elements have been associated with tissue which has a non-arterial blood supply, are regarded as a penumbra, or
image elements with image values lying within the specified range of variation in all relevant images are regarded as a parenchyma.

20. The method as claimed in claim 6, wherein the series of medical X-ray based image recordings includes fewer than five successive recordings.

21. The method as claimed in claim 7, wherein the relevant image values are read from relevant images other than the maximum image.

22. The apparatus as claimed in claim 11, further comprising:
a filter unit configured to segment bones and vessels in image recordings,
mask segmented regions, and
filter regions other than the segmented regions, wherein
the filter unit includes a bilateral filter.

23. A control device to control a medical-engineering imaging system, the control device configured to carry out the method of claim 1.
